(19) United States
(12) Patent Application Publication
(10) Pub. No.: US 2022/0143349 A1
(43) Pub. Date: May 12, 2022

(54) RESPIRATORY MASK

(71) Applicant: Wade Lamont Yoder, Fort Valley, GA (US)

(72) Inventor: Wade Lamont Yoder, Fort Valley, GA (US)

(21) Appl. No.: 16/949,610

(22) Filed: Nov. 6, 2020

(51) Int. Cl. A61M 16/06; A61M 16/10

(52) U.S. Cl. CPC A61M 16/0683; A61M 16/105; A61M 2205/02; A61M 2202/0225; A61M 16/0616

(57) ABSTRACT

A respiratory protective mask that prevents carbon dioxide rebreathing is disclosed. The respiratory mask includes a filter element that is dimensioned to cover an opening of a wearer's mouth. A cup like element is configured to rest against and retain the filter element against a wearer's chin. A top extent of the filter element extends laterally across a wearer's lip between a wearer's nose and the opening of the wearer's mouth, when worn. An arm of the filter element is defined by a convergence of the top extent and a lower extent of the filter element. The lower extent that converges upwardly from the cup like element towards a lateral end of the filter element subjacent to a wearer's ear, when worn. A retaining element is attached at the lateral end of the filter element and is configured to removably secure the filter element to cover the wearer's mouth.

### BACKGROUND OF THE INVENTION

The present invention relates to personal protective equipment, and more particularly to personal respiratory face masks.

Traditional personal protective masks are dimensioned to cover the user's nasal and oral cavities. Wearing these traditional respiratory protective masks may cause a user's blood carbon dioxide level to increase due to rebreathing of exhaled air. Studies indicate that mask wearing is primarily to prevent an infected carrier from infecting others due to exhaled droplets, rather than primarily protecting the wearer from airborne infection. Carbon dioxide from air that has already been breathed can be unhealthy healthy and forcing people to do this solely to enforce a mask policy can be deleterious to the wearer.

As can be seen, there is a need for an improved respiratory mask that assists users breathe unrestricted through the nasal passages, while still protecting the public from the primary source of respiratory droplets (from the wearers mouth).

### SUMMARY OF THE INVENTION

In one aspect of the present invention, a respiratory mask is disclosed. The respiratory mask includes a filter element that is dimensioned to cover an opening of a wearer's mouth. A cup like element is configured to rest against and retain the filter element against a wearer's chin. A top extent of the filter element extends laterally across a wearer's lip between a wearer's nose and the opening of the wearer's mouth, when worn. An arm of the filter element is defined by a convergence of the top extent and a lower extent of the filter element. The lower extent that converges upwardly from the cup like element towards a lateral end of the filter element subjacent to a wearer's ear, when worn. A retaining element is attached at the lateral end of the filter element. The retaining element is configured to removably secure the filter element to cover the opening of the wearer's mouth.

In some embodiments, the filter element has a bilateral symmetry about a medial axis of the filter element.

In some embodiments, the retaining element includes an elastic band attached from the lateral end of the filter element. The elastic band may have an adjustment means for setting a desired tension of the filter element across the opening of the wearer's mouth. The adjustment means may include a buckle.

In other embodiments, the retaining element may be a first tie string attached to the end of the filter element. A second tie string attached along the top extent of the filter element and medially spaced apart from the end of the filter element. The first tie element and the second tie element are formed of an elastic band.

In yet other embodiments, the retaining element includes an ear loop. An upper end of the ear loop is attached to the top extent of the filter element and medially spaced apart from the end of the filter element. A lower end of the ear loop is attached to the end of the filter element.

The filter element may be made from a woven or a non-woven fabric.

These and other features, aspects and advantages of the present invention will become better understood with reference to the following drawings, description and claims.

### BRIEF DESCRIPTION OF THE DRAWINGS

FIG. 1: Shows a bandanna style iteration of the respiratory mask in-use.

FIG. 2: Shows a string style iteration of the respiratory mask in-use.

FIG. 3: Shows an ear loop style iteration of the respiratory mask in-use.

### DETAILED DESCRIPTION

The following detailed description is of the best currently contemplated modes of carrying out exemplary embodiments of the invention. The description is not to be taken in a limiting sense, but is made merely for the purpose of illustrating the general principles of the invention.

Broadly, embodiments of the present invention provides an improved respiratory mask that limits the incidence of carbon dioxide rebreathing by the wearer. The present invention provides a mouth only mask that leaves the nose exposed and free to breathe new air. The mouth covering catches respiratory droplets from the primary source of respiratory droplet transmission, the mouth.

Other available respiratory masks are constructed as if the air is bad around the user. However, the mouth only mask according to the present invention will catch respiratory droplets exhaled through the wearer's mouth, while still allowing the user to have the option to breathe clean fresh air through their nasal passages rather than rebreathed, high carbon dioxide are, resulting in a lower carbon dioxide intake by the wearer. The mask of the present invention helps users breathe unrestricted through the nasal passages, while still protecting the public from the primary source of respiratory droplets (from the wearers mouth).

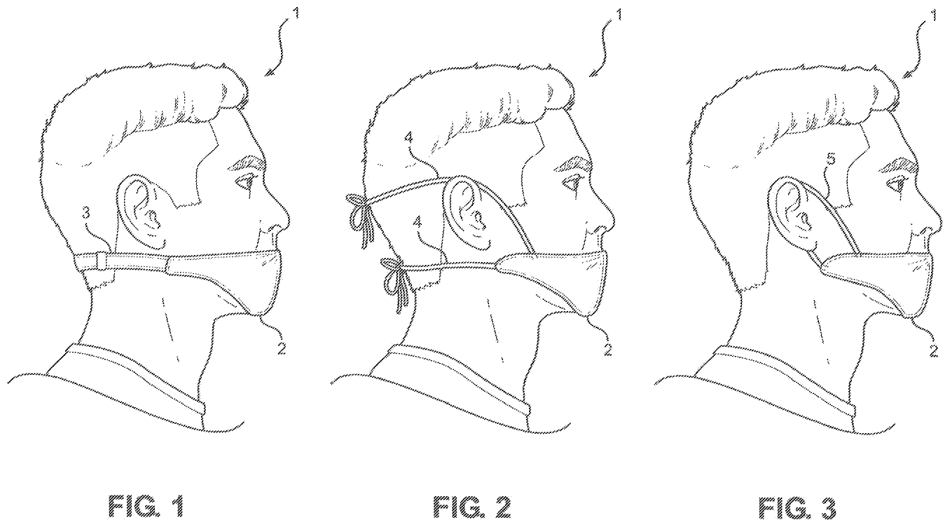

As seen in reference to the drawings of FIGS. 1-3, the respiratory mask 1 of the present invention includes a filter element 2 that is dimensioned to cover the oral opening of the mouth and has a cup like element to rest against and retain the filter element 2 against the wearer's chin. A top extent of the filter element 2 extends laterally across the wearer's lip between the wearer's nose and the opening of the mouth. An arm of the filter element 2 extends laterally with a lower extent of the filter element 2 extending upwardly from the cup like element and converging towards a lateral end of the filter element 2, and subjacent to the wearer's ear. The filter element 2 of the respiratory mask 1 has a bilateral symmetry about a medial axis of the filter element 2.

As seen in reference to FIG. 1, the filter element 2 is secured to the wearer's head by an elastic band 3 extending from the lateral end of the filter element 2. The elastic band 3 may have a buckle or adjustment means for setting a desired tension of the filter element across the wearer's mouth.

In first alternative embodiment, shown in reference to FIG. 2, a first tie string is attached to the end of the filter element with a second tie element attached along the top extent of the filter element 2 and medially spaced apart from the end of the filter element 2. The first tie element and the second tie element may be formed of a string or an elastic band to adjust tension of the filter element 2 to the wearer's mouth.

A second alternative embodiment is show in reference to FIG. 3. In this embodiment an ear loop 5 is provided. An upper end of the ear loop is attached to the top extend of the filter element 2 and medially spaced apart from the end of the filter element 2. A lower end of the ear loop 5 is attached to the end of the filter element 2.

Securement of the retaining elements 3, 4, and 5 at the end of the filter element 2 ensures that the filter element 2 is drawn laterally across the wearer's mask. Securement of the retaining elements 4 and 5 along the top extent of the filter element 2 and medially spaced apart from the end of the filter element 2 assists with preventing the filter element 2 from drooping on the wearer's face.

The filter element 2 may be formed of a suitable filtration medium indicated by a prevailing pathogen. The filter element 2 may be formed of either a woven or non-woven material. The filter element may include a stitch, binding, or a bias around the peripheral extents of the filter element 2.

The mask part that covers the mouth and chin is held in position by loops that go around the ear or fasten with a neck strap that wraps around the back of head/neck

The filter element may be sewn from a fabric designed to fit comfortably around the mouth and chin. The cup element needs to come far enough under the chin to cup the filter element 2 in position to catch respiratory droplets emitted as the wearer exhales through the mouth. The mask can be attached to the face by fasteners that either loop around ears or wrap around to the back of head/neck.

Both the mouth/chin piece and the way it fastens are equally important. The wearer may have this on for long periods, so whether it is secured by loops that go around the ear or strap all the way around the back of the head, comfort is of utmost importance. The profile and fit of the filter element 2 allow for wearer comfort forget it is on his/her face.

The wearer would use it to cover their mouth while around others to protect others from the wearers respiratory droplets.

It should be understood, of course, that the foregoing relates to exemplary embodiments of the invention and that modifications may be made without departing from the spirit and scope of the invention as set forth in the following claims.

What is claimed is:

1. A respiratory mask, comprising:
a filter element that is dimensioned to cover an opening of a wearer's mouth, a cup like element configured to rest against and retain the filter element against a wearer's chin, a top extent of the filter element extends laterally across a wearer's lip between a wearer's nose and the opening of the wearer's mouth, when worn, an arm of the filter element is defined by a convergence of the top extent and a lower extent of the filter element, the lower extent that extends upwardly from the cup like element and converges towards a lateral end of the filter element subjacent to a wearer's ear, when worn; and
a retaining element attached at the lateral end of the filter element the retaining element configured to removably secure the filter element to cover the opening of the wearer's mouth.

2. The respiratory mask of claim 1, wherein the filter element has a bilateral symmetry about a medial axis of the filter element.

3. The respiratory mask of claim 2, the retaining element comprising:
an elastic band attached from the lateral end of the filter element.

4. The respiratory mask of claim 3, wherein the elastic band has an adjustment means for setting a desired tension of the filter element across the opening of the wearer's mouth.

5. The respiratory mask of claim 4, wherein the adjustment means comprises:
a buckle.

6. The respiratory mask of claim 2, wherein the retaining element comprises:
a first tie string attached to the end of the filter element.

7. The respiratory mask of claim 6, further comprising:
a second tie element attached along the top extent of the filter element and medially spaced apart from the end of the filter element.

8. The respiratory mask of claim 7, wherein the first tie element and the second tie element are formed of an elastic band.

9. The respiratory mask of claim 2, wherein the retaining element comprises:
an ear loop, an upper end of the ear loop is attached to the top extent of the filter element and medially spaced apart from the end of the filter element; and
a lower end of the ear loop is attached to the end of the filter element.

10. The respiratory mask of claim 1, wherein the filter element is made from a woven or a non-woven fabric.
